(19) United States
(12) Patent Application Publication
(10) Pub. No.: US 2014/0172047 A1
(43) Pub. Date: Jun. 19, 2014

(54) IMPLANTABLE PULSE GENERATOR FOR STIMULATION OF A NEUROLOGICAL CELLULAR MASS

(71) Applicants: Hartmut Spitaels, Lubbeek (BE); Koenraad F. Van Schuylenbergh, Vorselaar (BE); Thomas Kaiser, Antwerpen (BE)

(72) Inventors: Hartmut Spitaels, Lubbeek (BE); Koenraad F. Van Schuylenbergh, Vorselaar (BE); Thomas Kaiser, Antwerpen (BE)

(73) Assignee: SYNAPTIX N.V., Niel (BE)

(21) Appl. No.: 14/119,168

(22) PCT Filed: Dec. 16, 2010
(86) PCT No.: PCT/EP2010/069949

§ 371 Date: Nov. 20, 2013

Related U.S. Application Data
(60) Provisional application No. 61/288,051, filed on Dec. 18, 2009.

(30) Foreign Application Priority Data
Dec. 18, 2009 (EP) 09179845.4

(51) Int. Cl. A61N 1/375; A61N 1/372

(52) U.S. Cl. CPC A61N 1/375; A61N 1/37211
USPC 607/60; 607/72

(57) ABSTRACT

The invention relates to an implantable pulse generator (100), IPG, for stimulation of a neurological cellular mass comprising a casing (2) that at least partially encloses the pulse generating module (PGM) (4) and that is transparent to radio-frequency electromagnetic fields, or wherein the pulse generating module (4) includes a controller circuit (18) provided as two or more circuit boards (20, 22), co-operatively connected, one such circuit board being an interface circuit board, where at least one component of the controller circuit (8) is located on the interface circuit board (20), and feed through wires for connector block (6) are connected thereto (20), and one of the opposing surfaces of the interface circuit board (20) is aligned over apertures (48, 50, 52) in the PGM housing for the feed through wires.

### FIELD OF THE INVENTION

The present invention relates to medical devices used to electrically stimulate a neurological cellular mass, in particular, neural tissue of the brain, and more specifically to an implantable pulse generator therefor.

### BACKGROUND OF THE INVENTION

Electrical neurostimulation techniques are used to treat a variety of conditions, such as coronary disorders, gastric dysfunction and neurological conditions. An example of electrical neruostimulation is Deep Brain Stimulation (DBS) which is a technique that may be used as a part of a treatment for various neurological disorders, such as Parkinson's Disease, Huntington's disease, dystonia, and epilepsy, among others. In DBS, one or more probes is implanted into the neural tissue of the brain to administer electric pulses that have the effect of reducing the symptoms. A particular disorder will be associated with a particular region of the brain, therefore, stimulation needs to be site specific. Although not fully understood, DBS is becoming a more widely accepted treatment, as an alternative to or to complement drug therapy. Surgical techniques for both probe (lead) and pulse generator implantation are becoming standardized. Various implantable devices are currently available, an example of such a device is the Active Therapy System sold by Medtronic, Inc. of Minneapolis, Minn. (www.medtronic.com/physician/activa/implantable.html).

The pulses are generated by an implantable pulse generator (IPG) that is typically implanted subcutaneously in the thoracic region of the subject, and electrical pulses generated by the IPG are conducted via subcutaneous extension wires to leads terminating in electrical contacts which stimulate the neural tissue.

These IPG devices, however, are generally large, owing to the requirement for a battery pack with sufficient power output, depth of discharge and lifespan that avoids the need to replace the battery frequently. Although the average lifespan of an implant's battery is 3- to 5-year, another surgical intervention is required to replace the whole device, which can be costly and inconvenient. A more powerful battery will increase battery lifespan, however, such battery is larger and heavier, and increases the overall size of the IPG. When the IPG exceeds a certain dimension, it becomes technically challenging to implant without forming a protuberance at the site of implantation that can be visible, and, moreover, without subjecting the patient to discomfort due to its size and weight implanted. Thus, it would be advantageous to provide an IPG which reduces the intrusive appearance of the implant, and yet does not compromise on battery life or performance.

### SUMMARY OF THE INVENTION

One embodiment of the invention relates to an implantable pulse generator (100), IPG, for stimulation of a neurological cellular mass comprising:
a pulse generating module (4), PGM, provided in an hermetically sealed housing (40),
an electrical connector block (6) for electrically connecting output from the PGM (4) to one or more leads,
a first aerial (8) for wireless exchange of data with the PGM (4),
a second aerial (10) for wireless receipt of inductive electrical energy to the PGM, and
a casing (2) that at least partially encloses the PGM and that is transparent to radio-frequency electromagnetic fields, wherein the connector block (6) and aerials (10, 8) are contained within the material of said casing (2).

2. IPG according to claim 1, wherein the second aerial (10) is coiled around the peripheral edge (60) of the housing (40) of the PGM (4).

Another embodiment of the invention relates an IPG as described above, wherein the coil of the second aerial (10) is situated between one fictive plane (100), extending from an upper (56) surface of the housing (40) exterior and a second fictive plane (102) extending from a lower (58) surface of the housing (40) exterior.

Another embodiment of the invention relates an IPG as described above, wherein the casing (2) is substantially formed from silicone rubber or epoxy resin.

Another embodiment of the invention relates an implantable pulse generator (100), IPG, for stimulation of a neurological cellular mass comprising:
an hermetically sealed pulse generating module (4), PGM,
an electrical connector block (6) for electrically connecting output from the PGM (4) to one or more leads, wherein the PGM module (4) is provided in an hermetically sealed housing (40) enclosing a chamber (42) in which electrical components of the PGM module (4) are disposed, the housing (40) comprises apertures (48, 50, 52) through which feed-though wires for the connector block (6) pass, the electrical components of the PGM module (4) include a controller circuit (18) which enable operation of the PGM, the controller circuit (18) is provided as two or more circuit boards (20, 22), co-operatively connected, at least one component of the controller circuit (8) is located on an interface circuit board (20), and feed through wires for connector block (6) are connected thereto (20), and one of the opposing surfaces of the interface circuit board (20) is aligned over said apertures (48, 50, 52).

Another embodiment of the invention relates an IPG as described above, wherein the interface circuit board (20) comprises one or more filter components, each connected to a feed through wire (80) for the connector block (6) configured to filter out electrical and/or electromagnetic interference.

Another embodiment of the invention relates an IPG as described above, wherein the interface circuit board (20) comprises one or more tuning components connected to the first and/or second aerial (10, 8) configured to tune the aerial (10, 8) to receive electromagnetic signals in a pre-determined frequency range.

Another embodiment of the invention relates an IPG as described above, wherein the interface circuit board (20) comprises one or more protective components connected to the first and/or second aerial (10, 8) configured to protect the PGM from voltage surges.

Another embodiment of the invention relates an IPG as described above, wherein:
the housing (40) comprises a plurality of grounding elements (84, 86, 88), electrically connected to said housing and projecting into the chamber (42),
one of the opposing surfaces of the interface circuit board (20) is located over at least one of the grounding elements (84, 86, 88)
the grounding elements are electrically connected to the interface circuit board (20).

Another embodiment of the invention relates an IPG as described above, wherein:
the interface circuit board (20) is mechanically attached to the housing (40) by a circumferential electrically conductive element between the housing (40) and the interface board (20) that electrically connects the interface board (20) to the housing (40).

Another embodiment of the invention relates an IPG as described above, wherein:
the interface board (20) is electrically connected to the housing (40) by means of electrically conductive adhesive or solder.

Another embodiment of the invention relates an IPG as described above, wherein the housing (40) comprises a two-piece assembly having a lid-part (92) and a body-part (94) with a reciprocating opening for the lid-part (92), which lid (92) is closed and sealed over the opening in the body-part (94) of the housing (40), wherein:
the apertures (48, 50, 52) for the feed-through wires for the aerials (1, 8) and connector block (6) are located in the lid-part (92), and
the interface circuit board (20) is mounted on the lid-part (92), such that it resides inside the chamber (42) of the housing.

Another embodiment of the invention relates a system comprising:
an IPG according to any of claims 1 to 12,
an external remote programming device (130), configured to wirelessly exchange data with the IPG (100) via the first aerial (8).

Another embodiment of the invention relates to a system as described above, further comprising a remote charging device (150) (150) adapted to inductively charge a rechargeable power source (16) of the IPG (100) though the second aerial (10).

### FIGURE LEGENDS

FIG. 1 Shows a schematic sectional view through an IPG of the invention, in which the controller circuitry is confined to a single circuit board.

FIG. 2 Shows a perspective view of a Pulse Generating module (PGM) of the invention, having apertures for feed-through wires.

FIG. 3 Shows a transverse cross-section through the PGM through the plane indicated 54 of FIG. 2.

FIG. 4 Shows a block-diagram of a possible layout of the circuitry of the device of the invention, in which the controller circuitry is confined to a single circuit board.

FIG. 5 Shows a schematic sectional view through an IPG of the invention, in which the controller disposed on two circuit boards, one an interface circuit board situated close to apertures in the housing for feed-through wires.

FIG. 6 Shows a block-diagram of a possible layout of the circuitry of the device of the invention, in which the controller components are split between two electrically connected circuit boards.

FIG. 7 Shows a perspective view of a PGM of FIG. 2, with a cross-sectional line indicated.

FIGS. 8 to 10 Show cross-sectional views of the apertured edge of a PGM of FIG. 7, along the cross-sectional line of FIG. 7, in which the interface circuit board is disposed with different configuration of components.

FIG. 11 Shows a perspective view of a PGM wherein the housing is formed from a lid and body, which view indicates a cross-sectional line.

FIGS. 12 to 14 Show cross-sectional views of the lid of the PGM of FIG. 11, along the cross-sectional line of FIG. 11, in which the interface circuit board is disposed with different configurations of components.

FIG. 15 Shows a cross-section through the PGM of the invention, along the cross-sectional line of FIG. 11, and the path of the parasitic induction loop.

FIG. 16 Shows a transverse cross-section through the PGM of the invention, and the placement of the wires of the second aerial.

FIG. 17 Shows a schematic of a two-component external remote programming device, together with an IPG of the invention.

FIG. 17 Shows a schematic of an integrated external remote programming device, together with an IPG of the invention.

FIG. 19 Shows a schematic of an external remote charging device, together with an IPG of the invention.

FIG. 20 Shows a schematic of an exemplary lead for use with the invention.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0)

Reference is made in the description below to the drawings which exemplify particular embodiments of the invention; they are not at all intended to be limiting. The skilled person may adapt the device and substituent components and features according to the common practices of the person skilled in the art.

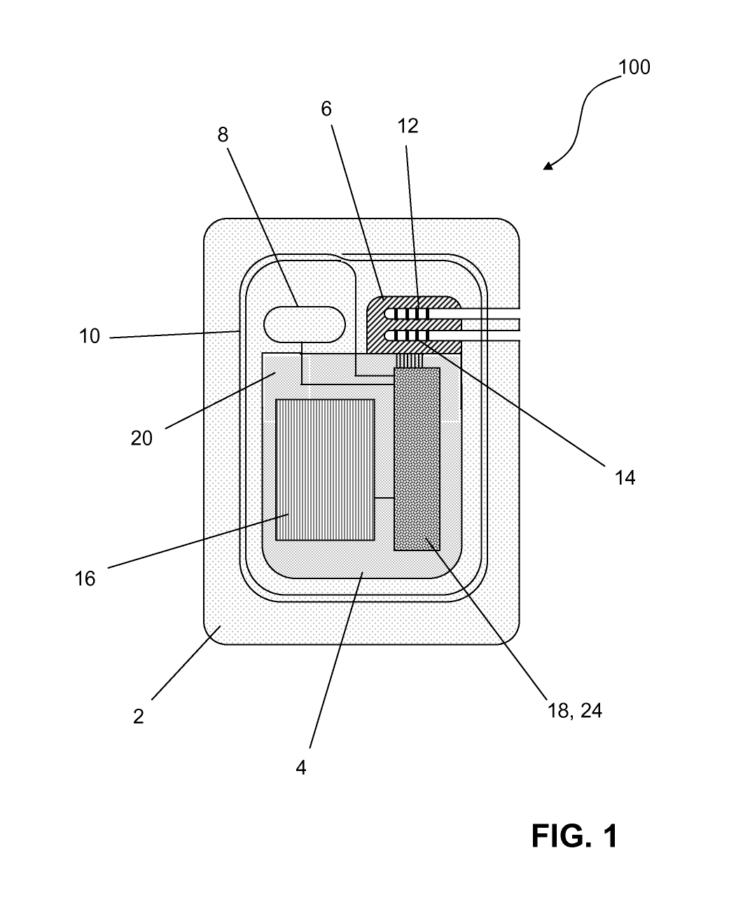

With reference to FIG. 1, the present invention concerns an implantable pulse generator 100, IPG, for stimulating a neurological cellular mass, in particular for deep brain stimulation comprising:
a pulse generating module 4, PGM, provided in an hermetically sealed housing 40,
an electrical connector block 6 for electrically connecting output from the PGM 4 to one or more leads,
a first aerial 8 for wireless exchange of data with the PGM,
a second aerial 10 for wireless receipt of inductive electrical energy to the PGM, and
a casing 2 that at least partially encloses the PGM and that is transparent to radio-frequency electromagnetic fields, RFEF, wherein the connector block 6 and aerials 10, 8 are contained within the material of said casing 2.

The aerials 10, 8 located in the RFEF-transparent casing 2 that at least partially encloses the PGM, facilitate the receipt of electrical energy and exchange of wireless signals. Advantageously, they are located outside the housing of the PGM having radio-shielding characteristics. Less energy is needed for the exchange of wireless signals, thereby reducing the battery drain. Moreover, longer communication distances are possible, allowing a deeper implantation of the device. In addition, charging times are reduced, and less wireless energy is passed through the skin.

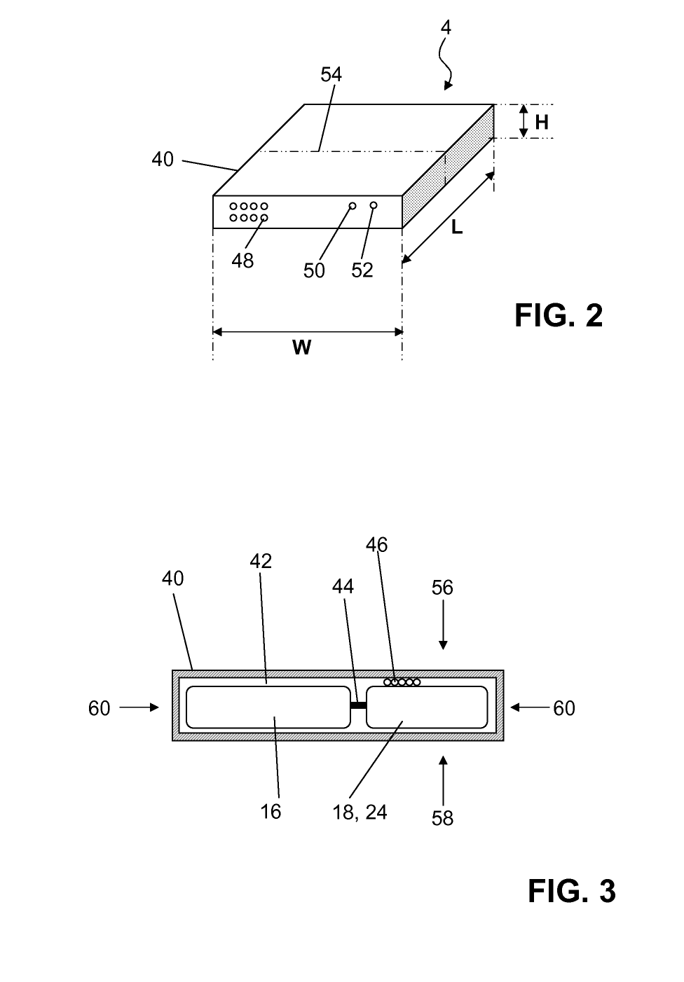

With reference to FIGS. 2 and 3, the PGM module 4 is provided in a housing 40 enclosing a chamber 42 into which components of the PGM module 4 are disposed (e.g. power source 16, controller 18 and connecting wires 44, 46 etc). The housing is formed from a fluid-impermeable substance such as titanium, or another suitable bio-compatible material. Feed-through wires for the aerials 10, 8 and connector block 6 pass through one or more apertures 48, 50, 52 in the wall of the housing. The housing 40 is hermetically sealed to prevent the permeance of bodily fluids such as blood and plasma into the chamber, or to prevent substances permeating from the housing such as battery fluid in the event of a leaking battery. Hermetic sealing of the apertures 48, 50, 52 may be achieved using any known technique, such as glass to metal pressure seals, or brazing of ceramic isolators. Such techniques incorporate an isolating component (e.g. glass or ceramic) between an electrical conductor and the aperture, while providing an intimate sealing between the aperture and the electrical conductor. Such intimate sealing can be provided with a pressure fitting or an hermetic chemical bonding.

The housing 40 is typically cuboid in shape, though other shapes are envisaged such as cylindrical, triangular or other irregular shape. In a preferred embodiment, the housing exterior has an upper 56 surface and lower 58 surface (FIG. 3), which surfaces are connected by a peripheral edge surface 60. When the housing is cuboid, as a general guidance, the upper and lower surfaces 56, 58 may have a length, L, of 5 cm, 6 cm, 7 cm, 8 cm, 9 cm or a value in the range between any two of the aforementioned values, and a width, W, of 5 cm, 6 cm, 7 cm, 8 cm, 9 cm or a value in the range between any two of the aforementioned values. The peripheral edge 60 may have a height, H, of 0.5 cm, 0.6 cm, 0.7 cm, 0.8 cm, 0.9 cm or 1 cm.

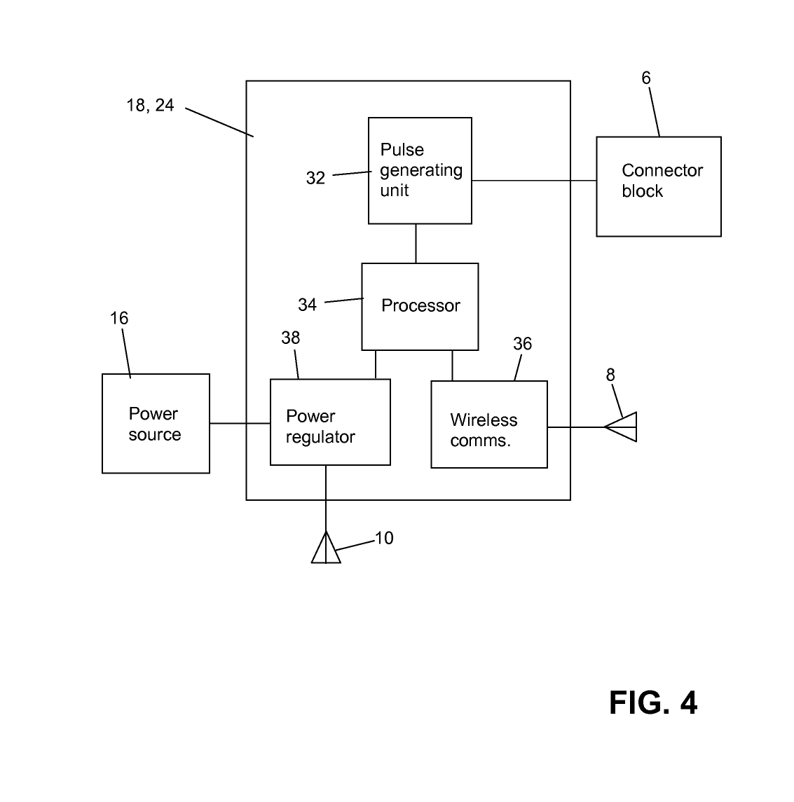

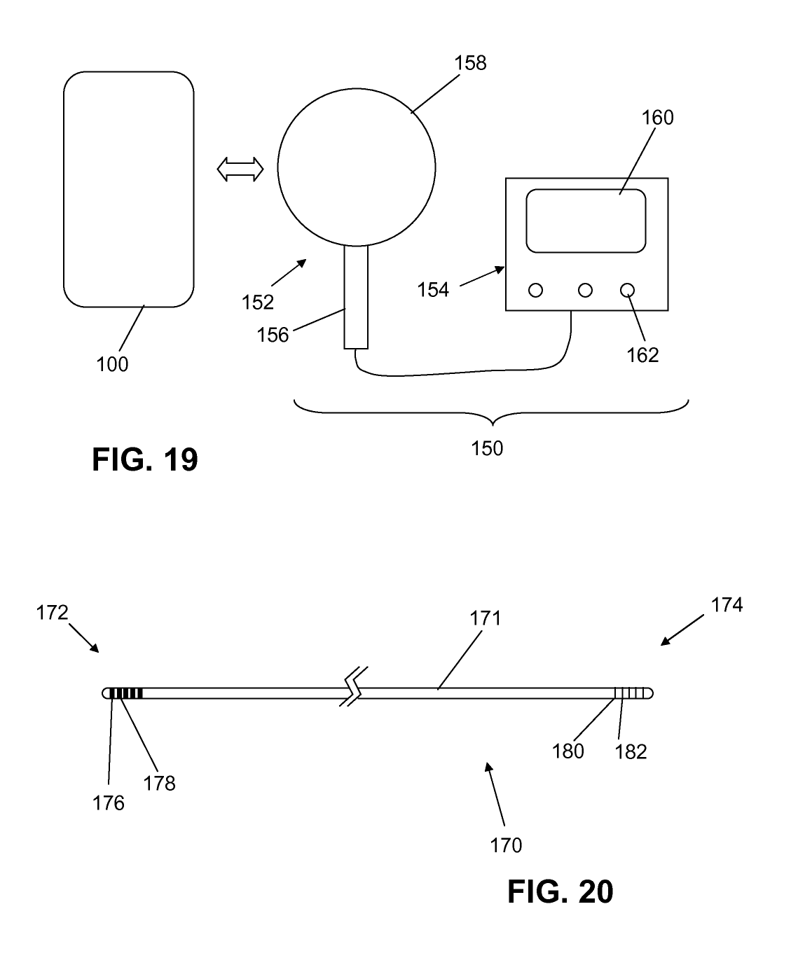

The PGM module 4 comprises a power source 16, that is typically a rechargeable battery which may be any of the art, including but not limited to those based on lead-acid, alkaline, Ni-iron, Ni-cadmium, NIH₂, NiMH, Ni-zinc, Li ion, Li polymer, LiFePO₄, Li sulfur, Nano Titanate, Thin film Li, ZnBr, V redox, NaS, Molten salt, Super iron or Silver zinc. The electrical energy is preferably supplied to the rechargeable battery inductively, that is to say, using inductive (magnetic) coupling, via the second aerial 10. The second aerial 10 is a coil that is inductively energized by a reciprocating induction coil of a remote charging device 150 (FIG. 19). The use of induction to transfer electrical power is well known in the art for example, from Schuder J. C., et al, “High-level electromagnetic energy transfer through a closed chest wall,” IRE Int. Conv. Record., vol. 9, pp. 119-126, 1961; Ko W. H., et al. “Design of radio-frequency powered coils for implant instruments,” Med. & Biol. Eng. & Comput., vol. 15, pp. 634-640, 1977; Donaldson N. de N. “Analysis of resonant coupled coils in the radio frequency transcutaneous links,” Med. & Biol. Eng. & Comput., vol. 21, pp. 612-627, 1983. The power source 16 is connected to a controller circuit 18, more specifically to a power regulator 38 (FIG. 4) incorporated in the controller, which is configured to convert inductive energy into usable electrical energy, and to control charging.

The second aerial 10 situated outside the chamber 42 of the housing 40 advantageously facilitates efficient transfer of inductive energy, compared with a second aerial 10 inside the housing chamber 42, especially when the housing 40 is formed from titanium. In the latter case, a loss of the magnetic field due to shielding of the housing prevents an efficient transfer. The consequence of the present configuration is reduced charging times, which is more convenient for the subject. Moreover, the implant may be more deeply located in the body without loss of the inductive link.

The PGM comprises a controller circuit 18 (FIGS. 3, 4) that enables the operation of the IPG. The controller circuit 18 incorporates one or more, preferably all, of the following elements:
a pulse generating unit 32 for generation of stimulating electrical pulses,
a wireless communications unit 36, for wirelessly exchanging data (i.e. transmitting and/or receiving data) through the first aerial with an external remote programming device,
a power regulator 38 adapted to convert inductive energy received through the second aerial 10 into electrical energy; and
a programmable processor 34 that controls the operation of the unit, including, the generation of the electrical pulses in the pulse generating unit 32.

Each of the elements is described in more detail below.

The power source 16 provides electrical power to a controller 18 that includes a pulse generating unit 32 configured to generate electrical pulses to the leads via the connector block 6. The pulse generating unit 32, is configured to generate a single sequence of pulses, or one or more sequences of pulses sequentially or simultaneously (see later). A sequence of pulses may be defined by the frequency of the electrical pulses, an amplitude of the electrical pulses, and pulse width of the electrical pulses.

The pulse generating unit may provide pulses using a variety of known techniques, for example, using a pulse generating circuit comprising a coupling capacitor that releases charge in response to a trigger signal provided by a digital controller/timer circuit, when an externally transmitted stimulation command is received, or when a response to other stored commands is received. By way of example, an output amplifier of the present invention may correspond generally to an output amplifier disclosed in U.S. Pat. No. 4,476,868 to Thompson, hereby incorporated by reference herein in its entirety.

According to one aspect of the invention, the pulse generating unit 32 may be controlled by a programmable processor 34 to operate so that it varies the rate at which it delivers stimulating pulses. The pulse generating unit 32 may further be controlled by a programmable processor 34 to operate so that it may vary the morphology of the stimulating pulses it delivers. Numerous features and functions not explicitly mentioned herein may be incorporated into the pulse generating unit while remaining within the scope of the present invention. Various embodiments of the present invention may be practiced in conjunction with one, two, three or more leads, or in conjunction with one, two, three, four or more electrodes. The pulse generating unit 32 may be single channel i.e. with one pulse generating circuit, capable of one output of electrical pulses, typically for a single or two contact lead.

The controller circuit 18 preferably includes a programmable processor 34. The programmable processor controls the triggering of the electrical pulses in the pulse generating unit 32. The processor is programmable, allowing the sequence of pulses and the destination electrical contacts to be adapted according to patient requirement. For example, the programmability may allow the surgeon to apply a generic sequence of pulses immediately after implantation surgery, which can later be fine-tuned to suit the patient's needs. The sequence of pulses may be based upon pulse parameters. The pulse parameters may specify the pulse morphology i.e. one or more of a frequency of the electrical pulses in a sequence, an amplitude of the electrical pulses in a sequence, a pulse width of the electrical pulses in a sequence, an on/off state of the electrical pulses, and an application location (i.e. to which electrodes) of the electrical pulses. The pulse parameters may further specify switching of a sequence of pulses in any distribution circuit, or, where there are a plurality of pulse-generating circuits, the sequence of pulses generated by each circuit.

The programmable processor 34 is connected to a wireless communications unit 36 (see below). The connection enables the programmable processor 34 to receive instructions and programs from the wireless communications unit 36 that have been transmitted from an external remote programming device 130. The connection also enables the programmable processor 34 to send information for transmission by the wireless communications unit 36, which information might concern status information, for instance, battery life, memory use, power consumption, electrical load, temperature etc.

The programmable processor 34 may optionally be connected to a power regulator 38 (see below). The connection may enable the programmable processor 34 to regulate power drawn from the power source 16 in the most efficient manner. The connection may also allow the programmable processor 34 to receive status information concerning the power source, which information can be transmitted externally by the wireless communications unit 36, upon demand. The processor 34 may also be used to control aspects of charging the power source by sending appropriate instructions to the power regulator 38.

In one embodiment of the invention, the controller 18 further includes a wireless communications unit 36, adapted to wirelessly exchange data (i.e. to transmit and/or receive data) with an external remote programming device. The data may concern the aforementioned pulse parameters which are received by the wireless communications unit 36. It may include information as to the status of the IPG 100, for example, battery status, temperature, internal diagnostics which data is transmitted from the IPG 100 and outside the body by the wireless communications unit 36.

The wireless communications unit 36, may utilise any wireless communication means including an RF (radio-frequency) link. It can adopt a technical standard for data transfer such as MICS (Medical implant Communications Service), Wi-fi, ZigBee or Bluetooth. The wireless communications unit 36, is connected to the first aerial 8 through which the data is wirelessly exchanged.

In another embodiment of the invention, the controller 18 further includes a power regulator 38 adapted to convert inductive energy received through the second aerial 10 into electrical energy; the electrical energy may be used to directly power the IPG, but more preferably, it is used to charge the rechargeable battery. The power regulator 38 converts energy received by inductively coupling the second aerial 10 with an external inductive loop applied over the skin in the region of the IPG 100. The use of induction to transfer electrical power is well known in the art as already described elsewhere herein.

The power regulator 38 may operate independently of the programmable processor 34. Alternatively, it may be connected to the programmable processor 34. The connection may enable the programmable processor 34 to regulate power drawn from the power source 16 in the most efficient manner. The connection may also allow the programmable processor 34 to receive status information concerning the power source, which information can be transmitted externally by the wireless communications unit 36. For example, when charging is complete, a signal may be sent by the power regulator 38 to the programmable processor 34 which in turn sends a stop signal for transmission by the a wireless communications unit 36. The processor 34 may also be used to control aspects of charging the power source, by sending appropriate instructions to the power regulator 38, for example, charging protocols.

According to one aspect of the invention, the controller 18 is provided as a single circuit board 24, containing electrical circuitry for processing, power regulation, wireless data exchange, pulse generation etc, as embodied, for instance, in FIGS. 1 and 4. The circuit board has two opposing surfaces, typically planar, the surfaces in essentially parallel alignment with the upper and lower surfaces 56, 58 of the housing 40 (FIG. 4).

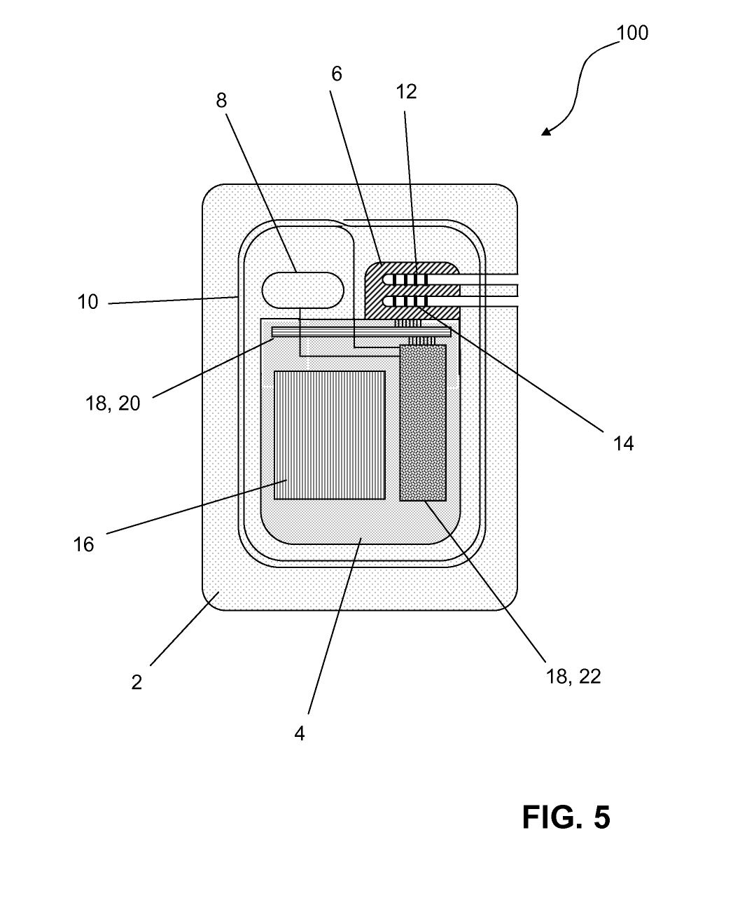

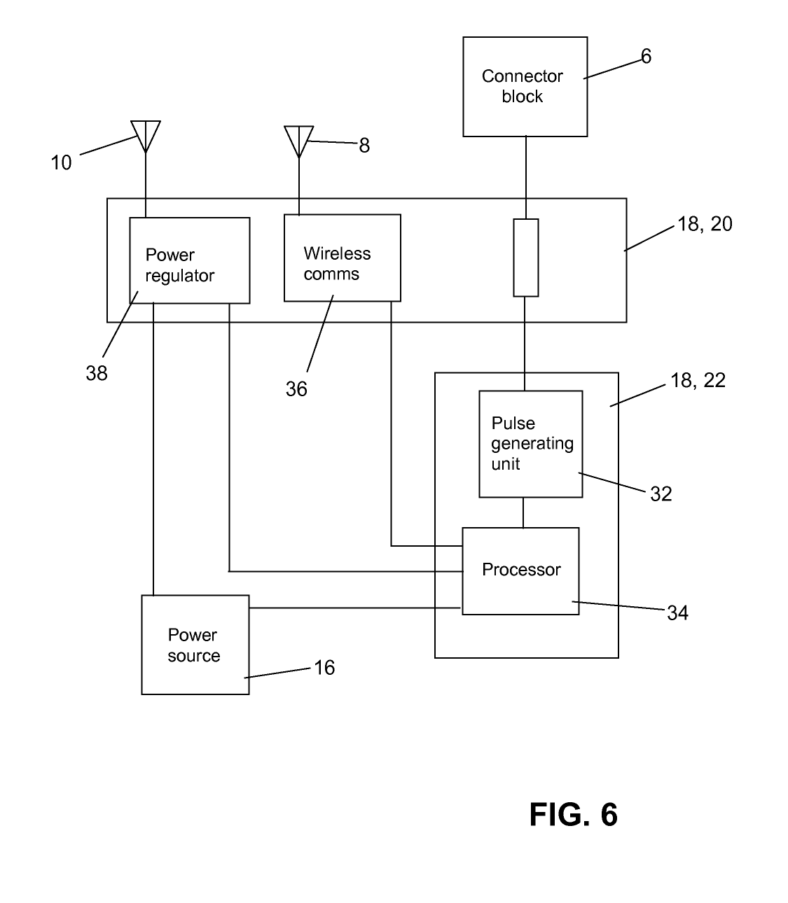

According to another embodiment of the invention, the controller 18 is provided as two or more circuit boards 20, 22, co-operatively connected, one such circuit board being an interface circuit board 20, wherein at least one component of the controller 18 is located on the interface circuit board 20, and feed through wires for the aerials 10, 8 and connector block 6 are connected thereto 20. Preferably, electrical components associated with the exchange of electrical energy or radio signals (RF components) are located on the interface circuit board 20. Preferably some, all or most of such components are provided on said interface circuit board 20. Examples of such components include a tuning component (78, 76) or a filter component (82), or electrical protection component; these are preferably only provided on the interface circuit board 20. This embodiment of the invention is shown, for example, in FIGS. 5 and 6. The interface circuit board 20 is located within the chamber 42 of the housing 40, proximal to the apertures 48, 50, 52 in the housing 40 through which the feed-though wires for the aerials 10, 8 and connector block 6 pass. The interface circuit board 20, in common with typical printed circuit boards, has two opposing surfaces, generally planar, either or both disposed with electrical components, and a peripheral edge; preferably, one surface of the interface circuit board 20 is aligned over said apertures 48, 50, 52. In other words, the plane formed by the apertures 48, 50, 52 is parallel to and overlaps the plane formed by one surface of the interface circuit board 20.

In order to minimize the length of feed-through wires, the interface circuit board 20 is positioned such that the apertures 48, 50, 52 in the housing 40 are aligned with reciprocating holes in the interface circuit board 20 for receiving and connecting the feed-through wires. In other words, a central axis at least one housing 40 aperture 48, 50, 52 is co-axial with a central axis of a reciprocating hole in the interface circuit board 20 for receiving the feed through wires. Preferably all the housing apertures and interface circuit board 20 holes are so-aligned. It will be understood that the central axes are tangential to the planar surfaces of the housing 40 or the interface circuit board 20. The arrangement avoids that the feed-through wires adopt a tortuous route i.e. inside the housing chamber 42, the feed-through wires have a linear and direct path to the interface circuit board 20. Minimising the length of the feed-through wires inside the housing 40 reduces potential electromagnetical interference of the RFEF with other electronic components inside the IPG. In other words, feed-through wires connected to aerials 8, 10 or a connector block 6 can transfer the RFEF through the aperture and have the potential to radiate the same RFEF energy inside the housing. i.e. they act as an extension of the aerials 8, 10 or connector block 6. Minimising the effective length of this extension avoids the aerial effect and reduces electromagnetical interference. Electronic components on the interface board 20 can filter and process relevant signals picked up by the aerials 8, 10 and/or lead 170 via the connector block 6 almost at the point where they enter the housing through the apertures. This arrangement minimizes the total conductive path of unfiltered and unprocessed RFEF inside the housing.

The other components (e.g. processor 34) of the controller 18 may be placed on a separate main circuit board 22, distal to said apertures 48, 50, 52 in the housing, but connected to the interface circuit board 20 using electrical conductors. The main circuit board 22, in common with typical printed circuit boards, has two opposing surfaces either or both disposed with electrical components, and a peripheral edge; preferably, one surface of the main circuit board 22 is aligned essentially parallel with the upper 56 or lower 58 surface of the housing 40. Typically, it will be perpendicular to the plane formed by the apertures 48, 50, 52.

Preferably the loop formed by a feed-through wire (70, 74, 72—FIG. 8, 9, 10, 12, 13, 14), the electrical connection to its tuning component (78, 76) or filter component (82), the tuning component 78, 76 or filter component itself 82, the interface circuit board 20, the grounding element 84, 86, 88 and the PGM housing 40 wall does not exceed 3 mm². By reducing the distance between RF components and the aerials 10, 8 i.e. locating the interface circuit board 20 close to the apertures 48, 50, 52, a better performance of the wireless communication link is achieved. This higher performance may be manifest in multiple aspects for instance, longer communication distance for any given form factor of the connector block, increased robustness of the link due to less susceptibility to interference, the antenna matching to operate in a human body is more reliable, increased omnidirectional functioning. This increased performance will ultimately lead to higher patient convenience and comfort.

Preferably, at least some or all the electrical components of the wireless communication device 36 are located on the interface circuit board 20. The components include at least tuning elements. This provides better performance in a cost effective way. Preferably, at least some or all the electrical components of the power regulator 38 are located on the interface circuit board 20. The components include at least filter elements. Preferably, at least some or all the signal feed-through wires for the connector block 6 are connected to the interface circuit board 20; said feed through wires may additionally be connected to filtering components on the circuit board 20 to filter out interference.

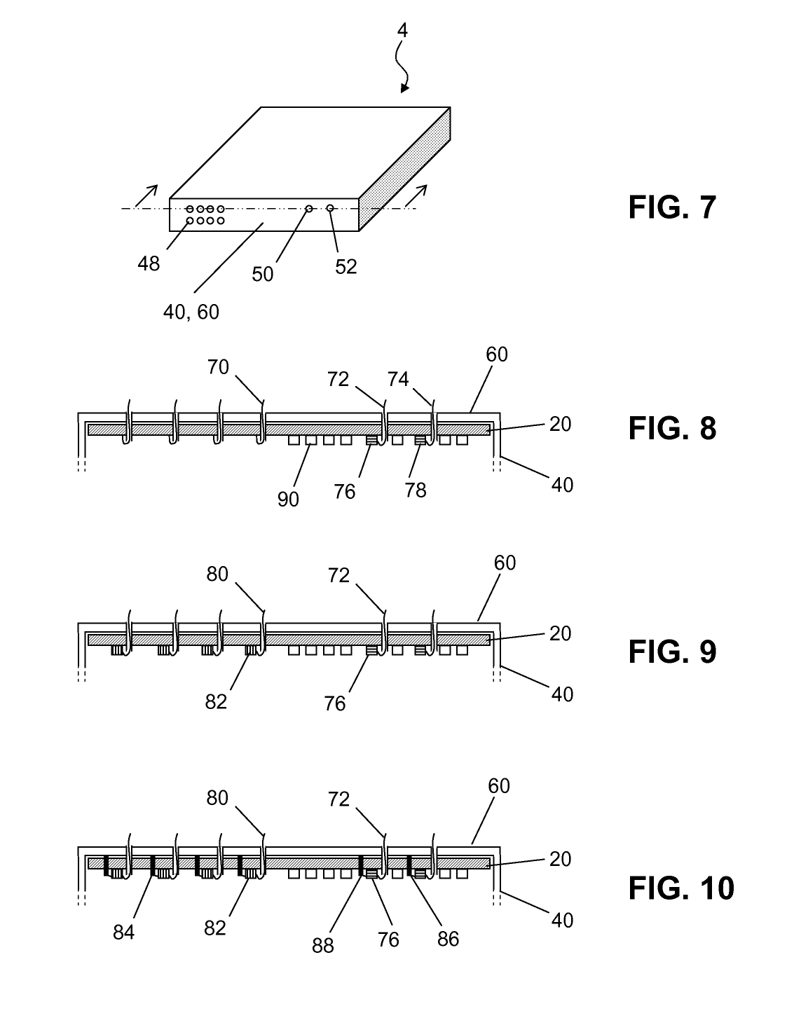

According to a specific embodiment of the invention, the apertures 48, 50, 52 are situated in a planar part of a peripheral edge surface 60 of the housing 40, and a surface of the interface circuit board 20 is aligned essentially parallel to said housing edge surface 60. This arrangement is depicted in FIGS. 7 to 10. In FIGS. 8 to 10, the interface circuit board 20 abuts and is parallel to the edge 60 of the housing 40, allowing feed-through wires 70, 74, 72 i.e. for the connector block, first and second aerials respectively to be minimised in length. While FIGS. 8 to 10, show one interface circuit board 20, it is within the scope of the invention that two or more (e.g. 2, 3, 4, 5, 6, 7) interface circuit boards 20 are stacked in parallel alignment in order to minimise distances from the feed-through wires to the electrical components.

According to the embodiment in FIG. 8, feed through wires 74, 72 for the first and/or second aerials respectively are each connected to a tuning component 78, 76 respectively on the interface circuit board 20. The tuning component 78, 76 preferably tunes the respective aerial to receive electromagnetic radiation centered around a pre-determined carrier frequency e.g. 401-401 MHz for the first 8 (data receiving) aerial. The reduction in distance between the aerials and tuning 78, 76 components greatly reduces interference, and provides, for instance, a more robust data link. Additional RF components 90 may be present on the interface circuit board 20 that may perform some of all of the tasks of the wireless communication unit and/or the power regulator.

The embodiment in FIG. 9, is similar to that in FIG. 8 with the addition of a feed through wire 80 for the connector block 6 connected to a filtering component 82 on the interface circuit board 20. The filtering component 82 removes signals of undesirable frequencies. The reduction in distance between the connector block 6 and filtering component 82 greatly reduces unwarranted interference that can sometimes have an influence when the subject is exposed to an environment of electromagnetic pollution, for example, when in close proximity to an activated cellular telephone. The most essential filtering components are capacitive elements configured as a low-pass filter system. The effectiveness of the filtering is correlated with the parasitic inductance of the filter system. This parasitic inductance is dominated by the geometrical area determined by the smallest current loop in the filter system. In the described arrangement of feed-through wires and filtering components, this current loop is minimized, hence also the parasitic impedance, and thus the configuration provides an optimal filtering performance. Current methods for reducing parasitic inductance use a technique of coaxial feed-trough filters in which electrical feed through wires are provided with a coaxial capacitive element embedded in the isolating ceramic element. However, a drawback of this technique is the higher cost and the limited flexibility of filter design.

Another possible function of filtering components is the elimination of unwanted signals from the pulse regulating unit towards the connector block and hence the neurological cellular mass. Typically low frequency or DC components of signals are considered harmful. Filtering elements configured as high-pass filters can effectively eliminate unwanted low-frequency content from the signal that is conducted from the pulse regulating unit to the cellular mass.

According to one embodiment of the invention, the interface circuit board 20 comprises one or more electrical protection components (not shown in the figures). These serve to protect the PGM, for instance, from voltage related surges, for example, a defibrillation pulse administered to the patient during heart recovery. The energy of this pulse is partially picked up by the lead 170 and could propagate through the connector block 6 into the PGM. Being located close to the feed through, the electrical protection components prevent damage before the surge reaches other more sensitive components. Such protective components could be for instance voltage limiting zener diodes electrically connected to the feed-through wires. Preferably some or all of the electrical protection components are provided on the interface circuit board 20.

The embodiment in FIG. 10, is similar to that in FIG. 8 with the addition of a plurality of grounding elements 84, 86, 88 connected to the housing 40. The grounding elements, which project into the housing chamber 42 are electrically conductive and act as grounding conductors, that can have an interference-shielding effect on the electrical components of the interface circuit board 20. This is effectively so when the housing acts as the electrical system ground. The grounding elements 84, 86, 88 may also serve as a mechanical attachment means to secure the interface circuit board 20 to the housing 40. One of the opposing surfaces of the interface circuit board 20 is located over at least one (e.g. 2, 3, 4, 5, 6, 7, 8, 9, 10), preferably all the grounding elements 84, 86, 88.

A grounding element 84, 86, 88 may be formed from the material of the housing 40, for instance, as a protruding extension thereof. Alternatively, it may be formed from a mass of adhesive-conductive material such as a conductive putty or adhesive, which secures the housing 40 to the interface circuit board 20.

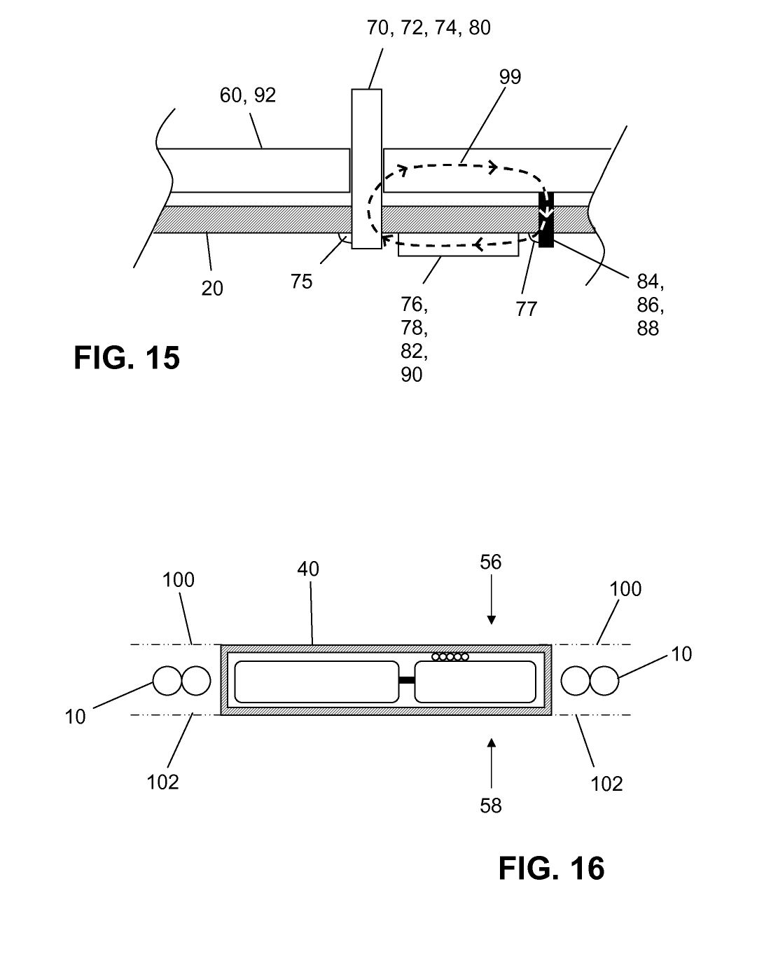

This arrangement of grounding elements in close proximity to the apertures and hence the feed through wires allows for a minimal conductive path for the RFEF signals through the filter, protective or tuning component to the system ground. The reduced path, and concomitantly reduced parasitic inductance is indicated in FIG. 15, which shows a feed through wire 70, 74, 72, 80 (e.g. for the connector block, first or second aerials) connected to an electronic component 76, 78, 82, 90 (e.g. tuning component, filtering component, protection component, other RF component) via the interface circuit board 20. It further shows a grounding element 84, 86, 88 connected to said RF component 76, 78, 82, 90 via the interface circuit board 20. Solder joints 75, 77 are indicated. The shortest current loop is depicted by the hatched line 99. It can be clearly deduced that the degree of parasitic inductance would be increased with increased distance between the electrically connected elements, which the present configuration avoids.

In addition or as an alternative, the interface circuit board (20) may be mechanically attached to the housing (40) by a closed loop (e.g. circumferential loop) of electrically conductive element between the housing (40) and the interface board (20). The electrically conductive element may be spring-loaded, which makes contact after being compressed.

In addition or as an alternative, the interface board (20) is electrically connected to the housing (40) by means of electrically conductive adhesive or solder.

According to one aspect of the invention, the housing 40 takes the form of a two-piece assembly comprising a lid-part and a body-part, wherein the interface circuit board 20 is mounted on the lid-part, which lid-part is closed and sealed over an opening in the body-part of the housing. The interface circuit board 20 is mounted on the surface of the lid-part that is closed over the opening. Using a lid-part as a chassis on which to assemble, mount and secure the interface circuit board 20 and feed through wires, considerably simplifies the production process, allowing access to the components for testing without undue hindrance. The interface circuit board 20 and lid-part combination combines the advantages of two conventional production techniques. The first conventional technique is the overmoulding of a silicone header onto the lid-part. The second conventional technique is the manufacturing of printed circuit boards. The grounding elements where present connect both methods in a unique way. The grounding protrusions elements create a solderable link between the interface circuit board 20 and the lid-part with its overmoulded header. This link creates a mechanical embedding of the lid-part, including antenna, on to the RF circuitry. It allows building the RF circuitry in the most cost-effective way without jeopardizing the performance. Without such a mechanical embedding the overall performance of the data and/or inductive link would be significantly reduced. It also benefits the overall immunity of the system to electromagnetic interference (EMI).

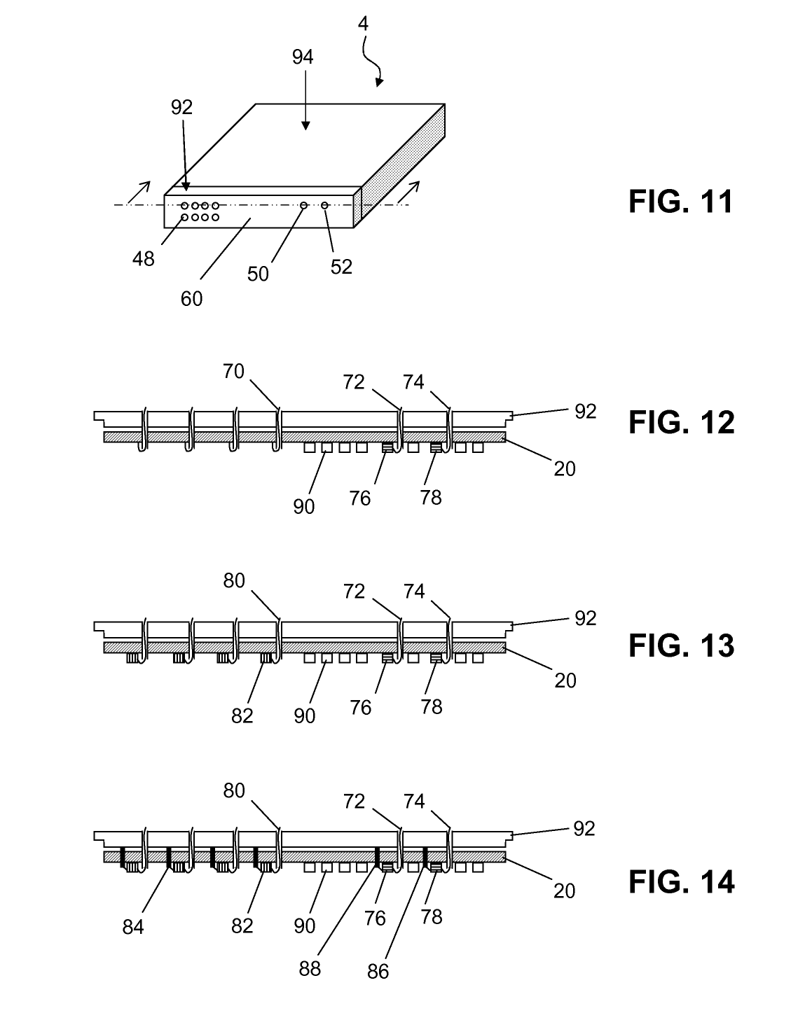

According to a specific embodiment of the invention, the lid-part 92 is formed from a planar peripheral edge surface 60 of the housing 40, and a surface of the interface circuit board 20 is aligned essentially parallel to said housing edge surface 60. Apertures 48, 50, 52 for feed though wires for the aerials 10, 8 and connector block 6 are situated in the lid-part 92 of the housing of the housing 40, and a surface of the interface circuit board 20 is aligned over said apertures 48, 50, 52. In other words, the plane formed by the apertures 48, 50, 52 in the lid is parallel to and overlaps the plane formed by one surface of the interface circuit board 20. This arrangement is depicted in FIGS. 11 to 14, which correspond to FIGS. 7 to 10 respectively, described earlier, with the exception that the housing planar edge is replaced with a lid 92 which is configured to close over an opening in the body 94 of the housing 40.

The lid 92 may be attached to the housing body 94 by any suitable technique including welding, adhesive, soldering or other that provides an hermetically sealed enclosure.

The IPG 100 comprises an electrical connector block 6 for electrically connecting the output from the pulse generating module 100 to one or more leads (also known as electrodes). The electrical connector block 6 is well known in the art, and any design may be employed by the instant IPG. As a general description, an electrical connector block 6 usually employs one or more cylindrical passages 12, 14 (FIGS. 1 and 5) each having a longitudinal axis, disposed with one or more contacts within the passage in electrical isolation arranged along the longitudinal axis of the cylinder, which electrical contacts are configured to establish electrical connection to a reciprocating cylindrical connector disposed with an equal number of contacts. The passage need not necessarily be cylindrical, though it is preferred. Each contact of the electrical connector is connected to circuitry in the PGM chamber via a plurality feed through wires as explained elsewhere.

The connector block 6 typically formed from a material different from the PGM housing and casing 2, for example, polypropylene, polycarbonate or polyurethane. The connector block 6, is contained within, preferably embedded in the casing 2 that is transparent to radio-frequency electromagnetic fields, RFEF, which casing at least partially surrounds the PGM.

The IPG 100 comprises a first aerial 8 for wireless exchange of data with the PGM. The aerial may be of any suitable configuration, depending on the strength of the signal and its frequency. As a guidance, the first aerial 8 is a loop of wire, optimized for the receipt and/or transmission of radio frequencies in the range 1 MHz to 3 GHz. Alternative configurations could be dipole or unipolar antennas. Typical loop sizes could range from 5 to 20 mm in diameter or typical antenna lengths could be between 5 mm and 50 mm.

The first aerial 8 is contained in an RFEF-transparent casing 2 that at least partially surrounds the PGM. Advantageously, the battery drain is reduced compared with aerials located in the PGM housing as typical housings in a conductive material (e.g. Titanium) act as a shield for RFEFs. Moreover, the IPG can be implanted more deeply into the body which reduces the possibility for visible lumps or discomfort in the subject.

The IPG 100 comprises a second aerial 10 for wireless receipt of inductive electrical energy to the PGM. The second aerial 10 may be of any suitable configuration. Generally, it is a coil, having a plane parallel to the one surfaces of the PGM 4 housing so that when the IPG is implanted, there is a natural and strong coupling to a reciprocating induction coil placed over the skin in essentially parallel alignment. A typical coil diameter would range between 30 mm and 100 mm or the circumferential area of the coil would range between 500 mm² and 3000 mm².

Preferably, the second aerial 10 is coiled around the peripheral edge 60 of the housing 40 of the PGM 4. Preferably, and with reference to FIG. 15, the coil of second aerial 10 is situated i.e. sandwiched between one fictive plane 100, extending from the upper 56 surface of the housing 40 exterior and a second fictive plane 102 extending from the lower 58 surface of the housing 40 exterior. The placement has been found to maximize the signal, compared with when the second aerial 10 is coiled in a region outside that delimited by the aforementioned planes.

The second aerial 10 is contained in an RFEF-transparent casing 2 that at least partially surrounds the PGM. Advantageously, less energy is required to charge the battery. Moreover, the IPG can be implanted more deeply into the body which reduces the possibility for visible lumps and/or discomfort in the subject.

One or more additional aerials may be arranged in the same RFEF-transparent casing tuned at frequencies different from the first or second aerial. A typical example would be an aerial for a separate wake-up transceiver circuit. Such a wake-up transceiver would typically operate with a minimal power consumption but would be limited in transfer rate or power transmission. The first and second aerials may or may not be capacitively coupled. Preferably, the first and second aerials are not capacitively coupled.

The IPG 100 comprises a casing 2 that is transparent to radio-frequency electromagnetic fields (RFEF). The casing 2 at least partially, preferably fully, encloses the PGM 4, the connector block 6 and aerials 10, 8. The contained elements are preferably embedded in the casing 2, though they may equally be contained within one or more void spaces formed within the casing 2. The casing 2 may be made for any suitable material that is biocompatible and having the aforementioned transparency to RFEF. An additional property of the casing material may be a shock absorbing property. Suitable materials include silicone rubber or epoxy resin. The casing preferably surrounds at least part of the PGM housing 40, preferably the entire PGM 4 housing 40. Where the casing 2 at least partially surrounds the PGM 4 housing 40, it may surround the peripheral edge 60; the upper 56 surface and/or lower 58 surface of the housing 40 may be at partially devoid of casing 2.

Cylindrical passages may be present in the RFEF-transparent casing 2 for connection to the leads; said passages are preferably co-axial with the one or more cylindrical passages 12, 14 (FIGS. 1 and 5) of the connector block 6. Advantageously, the RFEF-transparent casing 2 leads to significant improvement in signal gain through the aerials 10, 8, compared with the situation when the aerials enclosed within a titanium housing 40. The casing need not be hermetically sealed, since the permeation of moisture does not affect functioning of the aerials or connector block. Therefore, a more robust and efficient operation is achieved, that allows implantation deeper below the skin without loss of performance. Moreover, the shock absorbing property of the casing reduces impact damage to the IPG and causes less discomfort against adjoining tissue and bone structures.

A stimulation lead is well known in the art of stimulating a neurological cellular mass, in particular deep brain stimulation. With reference to FIG. 20, a lead 170 typically has an elongate body 171, a distal 174 and proximal end 176. A set of electrical contacts 180, 182 is provided at the distal end 174, which provide stimulation to the relevant target site. A set of slip ring contacts 176, 178 is provided at the proximal end 176, which either connect directly to the connector block 6 of the IPG, or to an extension lead. The number of electrical contacts may be any, for example, be in the range of 2, 3, 5, 6, 7, 8, 9, 10, 11, 12, 13, 14, 15, 16, 17, 18, 19, 20 or more, depending on the capability of the IPG and on the site of treatment and treatment regimen.

Electrical contacts are preferably arranged in an axial array, although other types of arrays may be employed. A lead 170 preferably range between about 10 cm and about 50 cm in length, and more particularly may be about 15 cm, about 20 cm, about 25 cm, about 30 cm, about 35 cm, about 40 cm or about 45 cm in length, depending on the location of the site to be stimulated and the distance of the IPG from the target. Other lead lengths such as less than about 10 cm and more than about 50 cm are also contemplated in the present invention. Some representative examples of leads 170 include MEDTRONIC nerve stimulation lead model numbers 3387, 3389 and 3391 as described in the MEDTRONIC Instruction for Use Manuals thereof, all hereby incorporated by reference herein, each in its respective entirety. Although FIG. 20 shows a certain lead configuration, other lead configuration as possible and contemplated in the present invention.

The IPG is provided for stimulation of a neurological cellular mass which can include a nerve cell, nerve bundles such as the acoustic nerve inside the cochlea and neurological tissue such as brain tissue and the spinal cord. The IPG can be used to treat a variety of medical disorders, depending primarily on the site at which the leads are implanted.

When the leads are implanted in the brain, the IPG can be employed to treat neurological conditions such as Parkinson's disease, Huntington's disease, dystonia and epilepsy, and other conditions still being researched.

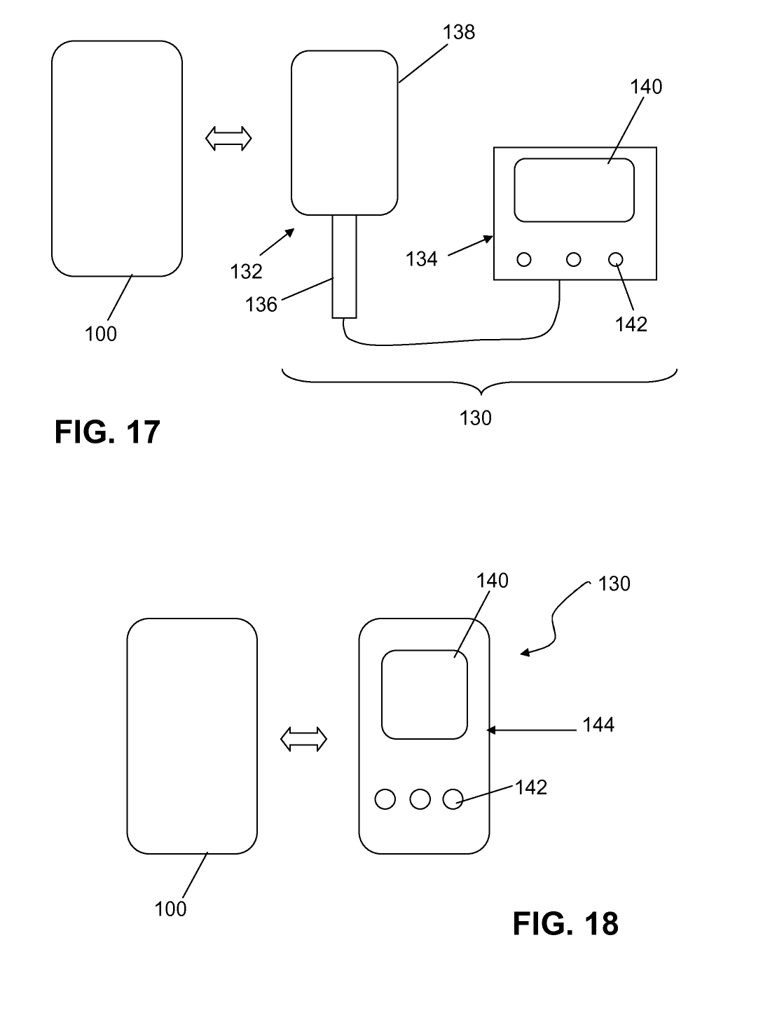

As mentioned elsewhere herein, a remote programming device 130 (FIG. 17) external to the patient's body is adapted, to wirelessly exchange data with the wireless communications unit 36. One embodiment of the invention is a system comprising an IPG 100 described herein and a remote programming device 130 external to the patient's body, adapted to wirelessly exchange data with the wireless communications unit 36. In particular, it may be adapted to transmit programming signals to the wireless communications unit 36 which are then provided to the programmable processor for adjusting the one or more pulse parameters. The remote programming device 130 may also be adapted to receive data from the IPG such as status information, temperature, battery condition etc. Remote programming devices 130 are known in general in the art, and typically comprise, as shown in FIG. 17, an antenna part 132 comprising an oblong-shaped housing 138 containing an antenna for placement over the implant 100 and a handle 136, and a transceiver part 134 comprising a screen 140 and controls 142 for the transmission and receipt of signals. While FIG. 17 shows the antenna part 132 and the transceiver part 134 as separate entities, it is within the scope of the invention that they are integrated into a single housing 144 as shown in FIG. 18. The transceiver part 134 may include a processor and user interface to assist with programming.

As mentioned elsewhere herein, a remote charging device 150 (FIG. 19) external to the patient's body is adapted to inductively charge the power source of the IPG. One embodiment of the invention is a system comprising an IPG 100 described herein and further comprising a remote charging device 150 external to the patient's body, adapted to inductively charge the power source of the IPG. Remote charging devices 150 are known in general in the art, and typically comprise, as shown in FIG. 19, an antenna part 132 comprising a disc-shaped housing 158 containing an inductive loop for placement over the implant 100 and a handle 156, and a controller 154 comprising a screen 160 and controls 162 for the transmission of coupling energy. The controller 154 may include a processor and user interface to assist with selecting a charging program. According to one embodiment of the invention, the remote programming device 130 incorporates a remote charging device 150.

1. An implantable pulse generator (IPG), for stimulation of a neurological cellular mass comprising:
an hermetically sealed pulse generating module (PGM), and
an electrical connector block configured to electrically connect output from the PGM to one or more leads, wherein the PGM is provided in an hermetically sealed housing enclosing a chamber in which electrical components of the PGM are disposed, the housing comprises apertures through which feed-though wires for the connector block pass, the electrical components of the PGM module include a controller circuit which enable operation of the PGM, the controller circuit is provided as two or more circuit boards, co-operatively connected, whereby one such circuit board is an interface circuit board, at least one component of the controller circuit is located on the interface circuit board, and feed through wires for connector block are connected thereto, and one of the opposing surfaces of the interface circuit board is aligned over said apertures.

2. IPG according to claim 2, wherein the interface circuit board comprises one or more filter components, each connected to a feed through wire for the connector block configured to filter out electrical and/or electromagnetic interference.

3. IPG according to claim 1, wherein the interface circuit board comprises one or more tuning components connected to the first and/or second aerial configured to tune the aerial to receive electromagnetic signals in a pre-determined frequency range.

4. IPG according to claim 1, wherein the interface circuit board comprises one or more protective components connected to the first and/or second aerial configured to protect the PGM from voltage surges.

5. IPG according to claim 1, wherein:
the housing comprises a plurality of grounding elements, electrically connected to said housing and projecting into the chamber,
one of the opposing surfaces of the interface circuit board (20) is located over at least one of the grounding elements,
the grounding elements are electrically connected to the interface circuit board.

6. IPG according to claim 1, wherein:
the interface circuit board is mechanically attached to the housing by a circumferential electrically conductive element between the housing and the interface board that electrically connects the interface board to the housing.

7. IPG according to claim 1, wherein:
the interface board is electrically connected to the housing by means of electrically conductive adhesive or solder.

8. IPG according to claim 1, wherein the housing comprises a two-piece assembly having a lid-part and a body-part with a reciprocating opening for the lid-part, which lid is closed and sealed over the opening in the body-part of the housing, wherein:
the apertures for the feed-through wires for the aerials and connector block are located in the lid-part (92), and
the interface circuit board is mounted on the lid-part, such that it resides inside the chamber of the housing.

9. An implantable pulse generator (IPG), for stimulation of a neurological cellular mass comprising:
a pulse generating module (PGM), provided in an hermetically sealed housing,
an electrical connector block configured to electrically connect output from the PGM to one or more leads,
a first aerial for wireless exchange of data with the PGM,
a second aerial for wireless receipt of inductive electrical energy to the PGM, and
a casing that at least partially encloses the PGM and that is transparent to radio-frequency electromagnetic fields, wherein the connector block and aerials are contained within the material of said casing.

10. IPG according to claim 9, wherein the second aerial is coiled around the peripheral edge of the housing of the PGM.

11. IPG according to claim 10, wherein the coil of the second aerial is situated between one fictive plane, extending from an upper surface of the housing exterior and a second fictive plane extending from a lower surface of the housing exterior.

12. IPG according to claim 9, wherein the casing is substantially formed from silicone rubber or epoxy resin.

13. A system comprising:
an IPG according to claim 1 or claim 9
an external remote programming device, configured to wirelessly exchange data with the I PG via the first aerial.

14. A system according to claim 13, further comprising a remote charging device adapted to inductively charge a rechargeable power source of the IPG though the second aerial.
